(19) United States
(12) Patent Application Publication
(10) Pub. No.: US 2011/0020909 A1
(43) Pub. Date: Jan. 27, 2011

(54) Methods Of Culturing Lawsonia Intracellularis

(75) Inventors: Jonathan Evans, Kalamazoo, MI (US); Connie Gebhart, Maple Grove, MN (US); Michael John Huether, Lincoln, NE (US); Rajendra Krishnan, Portage, MI (US); Gregory P. Nitzel, Mattawan, MI (US); Sharath K. Rai, Portage, MI (US); Catherine J. Streitzel, Kalamazoo, MI (US)

Correspondence Address: PFIZER INC;Mary J Hosley, 150 EAST 42ND STREET, MS: 150/02/E112, NEW YORK, NY, 10017-5612, US

(73) Assignee: Pfizer Inc., Madison, NJ (US)

(21) Appl. No.: 12/681,690

(22) PCT Filed: Oct. 14, 2008
(86) PCT No.: PCT/US08/79783

§ 371 Date: Oct. 5, 2010

Related U.S. Application Data
(60) Provisional application No. 60/998,742, filed on Oct. 12, 2007.

(51) Int. Cl. C12N 1/20

(52) U.S. Cl. 435/252.1

(57) ABSTRACT

The present invention relates generally to the growth of Lawsonia intracellularis in non-mammalian cells and the production of the bacteria on a large scale.

### FIELD OF THE INVENTION

The present invention relates generally to the growth of Lawsonia intracellularis in non-mammalian cells and the production of the bacteria on a large scale.

### BACKGROUND OF THE INVENTION

Porcine proliferative ileitis, sometimes referred to as porcine proliferative enteritis (PPE), is a major problem in the United States (US) swine industry. Proliferative ileitis is an intestinal disease complex of pigs characterized by crypt hyperplasia and by the presence of intracellular campylobacter-like organisms. Recognition of the disease has increased dramatically in the past ten years, with the incidence ranging as high as 20% and losses estimated at $50 million annually in the US alone. Especially alarming is the apparent increase in incidence among the seed stock industry. The disease has been found worldwide and usually affects post-weaning pigs between six and twenty weeks of age. The clinical signs of pigs affected with proliferative ileitis include intermittent diarrhea, anorexia, marked dullness and apathy, and a wasting syndrome. Death is not uncommon and is frequently associated with hemorrhage effects on intestines. Four different forms of the disease have been described, but the majority of the literature groups the lesions into two forms, acute and chronic, sometimes referred to as necrotic. Effective proliferative ileitis control measures have been limited. A basic trial-and-error therapeutic regimen, which includes the use of oral and parenteral broad-spectrum antibiotics, antihistamines, corticosteroids, nitroimidazole, and B vitamins, usually becomes quite costly and typically proves effective.

The presence of intracellular bacteria in the crypt of epithelium of afflicted animals confirms a bacterial etiology for the disease. Although bacteria isolated from such animals are morphologically similar to Campylobacter spp, hybridization studies and reproduction experiments using various Campylobacter strains have demonstrated that this organism is not the etiological agent. Joens and Glock (U.S. Pat. No. 5,610,059) describe and claim the isolation and characterization of a PPE organism and reproduction of the disease using the organism, which was previously referred to as PPE-causing agent, ileitis agent, IL-A, ATCC No. 55370, now known as Lawsonia intracellularis. The initial isolate was shown to reproduce the disease of proliferative ileitis. Since this initial report, at least four additional isolates have been obtained and shown to demonstrate the same growth characteristics as ATCC 55370, confirming that ATCC 55370 is the prototype organism.

International patent application PCT/US01/30284 describes proliferative ileitis vaccines prepared by growing L. intracellularis in a tissue culture selected from the group consisting of simian cells, murine cells, rat cells, canine cells, feline cells, hamster cells, human cells, equine cells, fish cells, bovine cells, and swine cells. L. intracellularis, a Gram negative obligate intracellular bacterium in the Desulfovibrio family, is difficult to isolate from field samples and grow in animal cells. There is, therefore, a need to grow large amounts of L. intracellularis in non-mammalian cells for use in vaccine development and production.

### SUMMARY OF THE INVENTION

The present inventors have developed methods for growing Lawsonia intracellularis in non-mammalian cells, especially insect cells and avian cells, and at a large scale useful for commercial production of vaccines.

According to the present invention, non-mammalian cells are planted in a vessel containing a suitable media, then inoculated with L. intracellularis. The cells are cultured under conditions identified herein appropriate for the growth and propagation of L. intracellularis. After harvesting, the cells are disrupted to release the L. intracellularis.

Suitable cells for use in the present methods include insect cells, Schneider cells, and avian cells. In a preferred embodiment, the cells are insect cells, such as Sf9 cells, SF21 cells, SF+ cells, Hi-Five cells, and insect larval cells. In another preferred embodiment, the cells are avian cells, particularly the CEV-1 cells.

The present invention has identified suitable densities of the cells seeded prior to inoculation, amounts of L. intracellularis in the inoculum, and multiplicities of infection. Inoculated cells can be cultured in an anchorage system or in suspension. The present invention has also identified desirable cell densities, depending upon whether the cells are cultured in an anchorage system or in suspension. Suitable culture media, temperature, atmospheric conditions, and periods of incubation are also described.

The methods of the present invention permit the propagation of Lawsonia intracellularis in non-mammalian cells and the production of the bacteria on a large scale for commercial manufacture of vaccines.

### BRIEF DESCRIPTION OF THE DRAWING

The file of this patent contains at least one drawing executed in color. Copies of this patent with color drawing(s) will be provided by the Patent and Trademark Office upon request and payment of the necessary fee.

FIG. 1 shows immunoperoxidose strain showing intracellular L. intracellularis in SF21 insect cells.

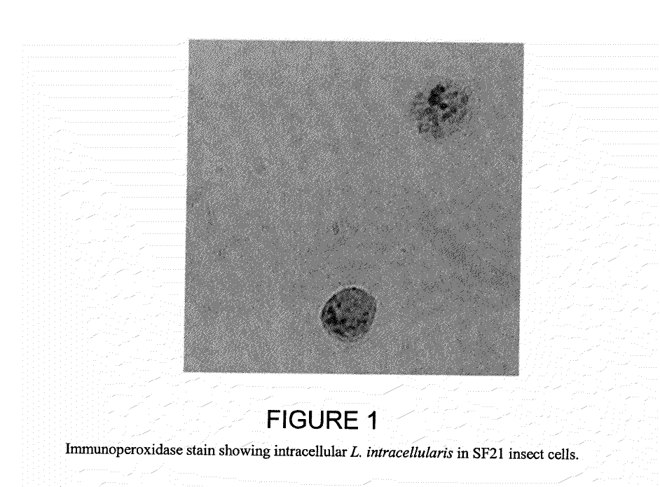

### DETAILED DESCRIPTION OF THE INVENTION

The present invention provides methods for the growth of virulent and/or avirulent Lawsonia intracellularis in non-mammalian cells and the production of the bacteria on a large scale. The methods of the present invention generally include the steps of 1) growing the Lawsonia intracellularis organism in a susceptible tissue culture utilizing a vessel containing media, and using a substrate for tissue attachment, or growing the L. intracellularis in suspensions of tissue culture cells; 2) harvesting the L. intracellularis by removing the grown L. intracellularis organisms from the tissue culture vessel; and 3) purifying the L. intracellularis organisms.

A significant impediment to the growth of Lawsonia intracellularis in non-mammalian and in particular insect cells is that such cells are non-natural hosts of such organisms, thus any growth, no less large-scale growth, would not be expected to be achievable. A further impediment faced by the present invention was that Lawsonia intracellularis typically grow in the 35° C.-39° C. range in the mammalian host. Insect cells, however, grow at 25° C.-29° C. and die quickly at 35° C.-39° C. The present invention, for the first time, provides methods for growth of virulent and/or avirulent Lawsonia intracellularis in non-mammalian cells and the production of the bacteria on a large scale. Moreover, while animal serum is generally used to propagate mammalian cells, and provide stabilizing factors for viruses and/or bacteria, in one embodiment, the present invention surprisingly achieves very high expression of Lawsonia intracellularis in insect cells without serum present. The achievement of growth and high levels of expression of Lawsonia intracellularis was unexpected and a remarkable achievement of the present invention.

In a further embodiment, the present invention provides growth of Lawsonia in avian cell lines.

### Definitions

In describing the present invention, the following definitions are used:

The terms “aerobic organism”, “aerobe”, and “aerophilic organism” refer to organisms that have an oxygen-based metabolism. The term “aerophilic condition” refers to conditions in which the oxygen concentration is about the same as that present in the atmosphere (i.e., about 20%).

The terms “anaerobic organism” and “anaerobe” refer to organisms that do not require oxygen for growth.

The term “anchorage system” and the like mean systems for culturing cells in which the cells form a sheet that is anchored to a vessel wall or a substrate, or the cells form a monolayer that is attached to a vessel or a substrate.

The term “continuous cell line” means a cell line which can be maintained in vitro for a limited number of cell divisions (up to approximately thirty) or indefinitely.

The terms “cultivation” and “culturing” mean the process of promoting the growth, reproduction, and/or proliferation of L. intracellularis organisms.

The term “fresh”, when referring to cells, means cells that have not been infected with L. intracellularis, and when referring to media means media that has not had cells in it.

The term “growth” means a produced increase in antigenic mass or cell density of the L. intracellularis in non-mammalian cells under appropriate temperature and temporal conditions. Growth can be measured by many art-recognized means including, but not limited to PCR, enzyme linked immunosorbant assay (ELISA), fluoresecent antibody staining (FA), and indirect fluorescent antibody staining (IFA).

The terms “large scale cultivation” and “commercial production” mean a level of cultivation of L. intracellularis greater than about 2 to 3 liters (L) and include production on a scale of at least 100 liters, and preferably 400 liters, or more preferably 1000 liters.

The term “matrix conditions” means the evaluation of a variety of conditions, including but not limited to, a full factorial of experiments that is conducted to elucidate an optimal method or a checkboard titration where one item is titrated on the y-axis and one item is titrated on the x-axis to reveal impact of the change.

The term “microaerophilic organism” refers to organisms that grow at low (subatmospheric) oxygen tensions. They require oxygen to survive, but require or can tolerate environments containing lower levels of oxygen than are present in the atmosphere. The term “microaerophilic condition” refers to conditions in which the oxygen concentration is lower than that present in the atmosphere (about 20%).

The term “microcarriers” means bead-like structures upon which the susceptible cells attach. They generally can be held in homogeneous suspension in stirred reactors.

The term “multiplicity of infection” (MOI) refers to a ratio of the number of organisms per cell, which details how much inoculum is going to be used in a given infection.

The term “passage” and the like mean the process of transferring a portion of a cell culture to fresh media.

The term “primary cell line” means a cell line which may be maintained in vitro for a limited period of time.

The term “suspension” means a system for culturing cells in which the cells are free-floating in the media as either single cells or as clumps of cells.

The term “spinner flask” means a flask or other container which employs a paddle, propeller, stir bar, or other means to agitate the culture and keep the cells contained therein in suspension.

The term “susceptible culture” means that the tissue culture has been specifically selected, cloned or established to grow a L. intracellularis organism and express the immunogens of the organism such that the immunogens are not modified or altered and an antigenic mass of the organism is produced.

The susceptible tissue culture useful for growing L. intracellularis can be either a primary or continuous cell line and can be established using a variety of non-mammalian cell types including, but not limited to, Schneider (Drosophila) cells, insect cells, insect larval cells, avian cells, avian embryo cells, and avian eggs. In one embodiment, the susceptible tissue culture is a culture of insect cells, such as Sf9, SF21, SF+ and Hi-Five cells. In a specific embodiment, the susceptible tissue culture is a culture of Sf9 cells. In another embodiment, the susceptible tissue culture is a culture of avian cells, for example, cells of the CEV-1 avian cell line.

A variety of matrix conditions can be used for growing the L. intracellularis organism in a susceptible tissue culture. Morphologically, the susceptible tissue culture may be grown as a suspension, as a cell sheet anchored to a vessel wall or a substrate, as a confluent monolayer attached to a vessel or substrate (microcarriers), or as semi-adherent cells wherein there is a mixed population of attached and suspension cells. The anchorage system may be fixed-bed, microfluidized bed, Wave reactor, stacked module, or air-lift. The vessel for growing a susceptible tissue culture can be, but is not limited to, flasks, T flasks, spinner flasks, roller bottles, cell trays, and bioreactors, containing media and using the vessel surface, beads, or other substrates for tissue culture attachment.

When growing the susceptible cells in suspension, the vessel can be, but is not limited to, flasks, T flasks, spinner flasks, Wave reactors, fermentors, and bioreactors, containing media. Vessels of any size in which the media can be mixed may be used, although the vessels are generally from about 50 ml to about 900 L in size. Preferably, about one-third of the vessel volume (50%) contains media, although other proportions of media to head space may be used. Susceptible cells can be grown on a small scale (e.g., a vessel containing about 50 ml to about 10 L of media), on a large scale (e.g., a vessel containing about 1,000 L to about 10,000 L of media), or on an intermediate scale (e.g., a vessel containing between about 10 L to about 1,000 L of media). In one embodiment, a vessel containing from about 100 L to about 600 L of media is used. In another embodiment, a vessel containing from about 100 L to about 400 L of media is used. In still another embodiment, a vessel containing about 150 L to about 250 L of media is used.

When growing the cells in suspension, cell density is generally in the range of about 100,000 to about 10,000,000 cells per ml. In one embodiment, cell density is in the range of about 200,000 to about 5,000,000 cells per ml. In another embodiment, cell density is in the range of about 500,000 to about 1,500,000 cells per ml. In suspension systems, cells can be mixed at a rate of about 25 to about 250 revolutions per minute. In one embodiment, cells are mixed at a rate of about 50 to about 150 revolutions per minute. In another embodiment, they are mixed at a rate of about 80 to about 120 revolutions per minute.

When growing the cells in an anchorage system, one form of vessels for culturing the cell lines and propagation of L. intracellularis is a stacked module system. The stacked modules can have a surface area of about 21,000 cm²to about 340,000 cm². Alternatively, other forms of vessels suitable for use include flasks, which may have a surface area of about 150 cm² to about 420 cm² and roller bottles which may have a surface area of about 1760 cm² but can range from about 850 cm² to about 4250 cm².

When growing the cells in an anchorage system, cell density is generally in the range of about 10,000 to about 1,000,000 cells per cm². In one embodiment, cell density is in the range of about 20,000 to about 500,000 cells per cm². In another embodiment, cell density is in the range of about 60,000 to about 250,000 cells per cm². In anchorage systems, roller bottles can be rotated at a rate of about 0.1 to about 100 revolutions per hour, while in cell trays and fixed-bed reactor the media is circulated through the vessel.

A suitable media formulation for culturing the cell lines and propagation of L. intracellularis can be any of the typical tissue culture media generally known to one skilled in the art for the type of cells being used. The media will generally include a nitrogen source, necessary growing factors for the chosen culture cells, and a carbon source, such as glucose or lactose. Some non-limiting examples of media formulations for culturing the cell lines include, but are not limited to, Ex-Cell™ 405, TNM-FH Insect Culture Medium (Gentaur Molecular Products, bvba), IPL-41 Insect Medium (Sigma-Aldrich Co.), Cellgro® Serum-Free Cell Culture Media (Mediatech, Inc.), and Dulbecco's modified eagle media (DMEM:F12 1:1) with L-Glutamine (Gibco® Cell Culture Systems, Invitrogen). In one embodiment, the cell culture media formulation is Ex-Cell™ 420 Serum-Free Medium for Isect Cells with L-glutamine (JRH Biosciences). In another embodiment, the cell culture media formulation is Dulbecco's modified eagle media (DMEM:F12 1:1) with L-Glutamine (Gibco® Cell Culture Systems, Invitrogen).

Cell culture media can be used in the absence or presence of animal derived components. An animal derived component that can be used is gamma-irradiated serum ranging from 0.5-10% final concentration. An example of such a component is Fetal Bovine Serum Sourced in USA gamma irradiated by SER-TAIN™ Process (JRH Biosciences). Generally, media that is animal-protein-free is preferable for insect cell cultures grown in suspension, while media that contains animal protein is preferable for insect cell cultures grown in an anchorage system.

The temperature for culturing the insect cell lines and propagation of L. intracellularis is generally in the range of about 20 to about 39 degrees C. In another embodiment, the temperature is in the range of about 23 to about 34 degrees C., and in still another embodiment, the range is from about 25 to about 29 degrees C. The temperature for culturing the avian cell lines and propagation of L. intracellularis is generally in the range of about 25 to about 45 degrees C. In another embodiment, the temperature is in the range of about 30 to about 40 degrees C., and in still another embodiment, the range is from about 35 to about 39 degrees C.

The atmospheric conditions for culturing the cell lines and propagation of L. intracellularis can be aerophilic or microaerophilic. In one embodiment, the cell lines are cultured in microaerophilic conditions comprising a mixture of about 10% hydrogen, about 10% CO₂ and about 80% nitrogen.

For the propagation of L. intracellularis, the cells are seeded into a chosen vessel. The vessel is generally seeded with between about 100,000 to about 10,000,000 cells per ml. In another embodiment, the vessel is generally seeded with between about 200,000 to about 5,000,000 cells per ml. Cells that have been passaged from 0 to about 20 times can be used for propagation of the L. intracellularis organism. In one embodiment, cells that have been passaged from about 10 to about 20 times are used for propagation.

A cell culture is initially inoculated with an inoculum containing L. intracellularis bacteria so as to infect the cells with the bacteria. The inoculum of L. intracellularis can be a pure culture obtained, for example, from American Type Culture Collection (ATCC, Rockville, Md.) deposit No. 55672, National Collection of Types Culture (NCTC, Colindale, London) deposit Nos. 12656 or 12657 (See U.S. Pat. No. 5,885,823) or from infected swine or other animals using isolation and purification techniques known to one skilled in the art. The amount of inoculum can be in the range of about 100 to about 1,000,000 Lawsonia copies per ml. In a specific embodiment, the amount of inoculum is in the range of about 200 to about 500,000 Lawsonia copies per ml. In another embodiment, the amount is in the range of about 400 to about 250,000 Lawsonia copies per ml.

The cell culture can be inoculated with the L. intracellularis organism at the time of planting the cells into the vessel or up to about five days after planting. In another embodiment, the cell culture is inoculated up to about 2 days after planting.

The multiplicity of infection (MOI) can be measured using standard techniques known to one skilled in the art, including fluorescent antibody staining (FA), indirect fluorescent antibody staining (IFA), polymerase chain reaction (PCR), and enzyme linked immunosorbant assay (ELISA). Two non-limiting examples of such techniques include qRT-PCR and TCID₅₀. The MOI for the propagation of L. intracellularis is generally in the range of about 0.000001 to about 10 using quantitative Reverse Transcriptase Polymerase Chain Reaction (qRT-PCR). In another embodiment, the MOI is in the range of about 0.00001 to about 10 using qRT-PCR. In still another embodiment, the MOI is in the range of about 0.0001 to about 10 using qRT-PCR.

The cell culture is allowed to incubate for a period of time (the incubation period) after infection with the L. intracellularis organism until the desired amount of growth of L. intracellularis has occurred. The incubation period can generally vary between about 5 and about 25 days after inoculating the cell culture with the L. intracellularis organism. The incubation period may also range from about 5 to about 15 days. In a specific embodiment for insect cells, the incubation period ranges from about 9 to about 13 days. In another embodiment for avian cells, the incubation period ranges from about 3 to about 13 days. The amount of growth can be measured using standard techniques known to one skilled in the art. Two examples of quantitative assays that can be used to assess the amount of growth include quantitative Reverse Transcriptase Polymerase Chain Reaction (qRT-PCR) and Tissue Culture Infective Dose 50 (TCID₅₀).

During the incubation period, the cell culture may be supplemented with fresh media, if desired. This may generally be done between about five to about nine days post-infection, or preferably, between about six to about eight days post-infection. The cell culture may be supplemented more than once during the incubation period, with between about three to about nine days between supplementations.

The incubation period may also include steps to scale up the process. For example, the cell culture can be seeded into a small infection vessel (e.g., about 5 L in size) and allowed to grow for a period of time (e.g., about one week). The culture can then be transferred to a larger vessel (e.g., about 30 L in size) and supplemented with fresh media. This process can be continued until the desired cell culture amount is achieved.

After the incubation period, a portion or all of the culture is harvested. The harvesting process requires removal of the fluids from the vessel. The fluids may contain cell debris or whole tissue culture cells in addition to the L. intracellularis. Harvesting is accomplished using standard techniques known to one skilled in the art, including but not limited to a freeze-thaw step, treatment with enzymes or detergents, or treatment with high pressures in order to break open the tissue culture cells to release the L. intracellularis organisms. Additionally, harvesting may include concentration using techniques known in the art such as centrifugation, continuous flow centrifugation, column chromatography, ultrafiltration, deadend depth filtration, or filtration with or with out cell debris in bulk product. For example, in one embodiment, the cells are harvested from the vessel, and PCR is used to quantitate the yield of the L. intracellularis bacteria.

In one example, the L. intracellularis bacteria are harvested by centrifuging the contents of all or a portion of the suspension to pellet the culture cells, resuspending the resulting cell pellets, and lysing the infected cells. If the cells are grown in an anchorage system, the cells are first disrupted to form a suspension. Typically, at least a portion of the contents is centrifuged at about 3000×gravity (g) for about 20 minutes in order to pellet the cells and bacteria. The pellet is then resuspended in, for example, fresh media or a sucrose-phosphate-glutamate (SPG) solution, and passed approximately four times through a 25 gauge needle in order to lyse the cells. If further purification is desired, the samples can be centrifuged at about 145×g for about five minutes to remove cellular nuclei and debris. The supernatant may then be centrifuged at about 3000×g for about twenty minutes and the resulting pellet resuspended in an appropriate diluent, such as fresh media or SPG with or without fetal bovine serum (to prepare harvested bacteria suitable for freezing or use as an inoculant) or growth media (to prepare harvested bacteria more suitable for passaging to fresh cells).

In another example, a continuous flow centrifuge may be used to collect the culture cells, which is then followed with a homogenization step to liberate the intracellular bacteria.

In one embodiment, the present invention is directed to vaccines which protect against proliferative ileitis which is caused by L. intracellularis sp. e.g. ATCC 55370 and all strains and mutants thereof which have similar immunogenic characteristics. By “immunogenic characteristics” is meant the ability to protect animals, e.g. pigs from proliferative ileitis. The contemplated vaccines include but are not limited to attenuated vaccines, inactivated vaccines, modified live vaccines, subunit vaccines and recombinant vaccines. The vaccine of the present invention is protective and/or therapeutic if it produces a high enough level of immunogen(s) and may include adjuants, stablizers, and/or excipients. Inactivation of L. intracellularis can be conventionally accomplished by treating the organism with BEI (binary ethyleneimine), BPL (beta-propiolactone), formalin, formaldehyde, heat or any other art known agents. Contemplated adjuvants include Amphigen®, Polygen®, Carbopul®, aluminum hydroxide, Freunds Complete Adjuvant, Freunds Incomplete Adjuvant, Iscoms or the like. Attenuated vaccines can be produced by serial passaging in tissue culture, for example. The vaccines can be administered intramuscularly, subcutaneously, intranasally, orally, intradermally or topically, for example.

The present invention also contemplates a diagnostic test for detecting the presence of proliferative ileitis in an animal. Accordingly, the invention provides monoclonal antibodies which can be utilized to diagnose or detect proliferative ileitis.

It is believed that one skilled in the art can, using the preceding descriptions, practice the present invention to its fullest extent. The present invention is further illustrated by the following detailed examples, which are provided for illustrative purposes only and are not to be construed as limiting the preceding disclosure in any way. The Lawsonia intracellularis employed in the examples that follow can be avirulent or virulent.

### Examples

### Example 1

PPE propagation experiment varying temperature and atmospheric conditions.

Purpose. The purpose of this experiment was to evaluate the growth of Lawsonia intracellularis using the Sf9 (spodoptera frugiperda) cell line at 27° centigrade (C.) (natural insect temperature) versus 37° C. and under CO₂ versus a specialty gas atmospheric conditions.

Materials and Methods.

| | | Part (item | |
|---|---|---|---|
| | Description | number) | Lot |
| Parent cell | Sf9 | na¹ | Pass 5 |
| Growth media | Ex-Cell ™ 420 | 14420-1000 M | 4N0352 |
| Live Lawsonia | Titer: | na¹ | na¹ |
| intracellularis | 2.5 dose/ml | | |

¹na = not applicable

Cell and Media Information. The cell culture was Sf9 cells (Gibco® Cell Culture Systems, Invitrogen, Carlsbad, Calif., USA). The growth media was Ex-Cell™ 420 Serum-Free Medium for Insect Cells with L-glutamine (JRH biosciences, Lenexa, Kans., USA; Catalog number 14420, item number 14420-1000M). The seed culture contained modified live, non-virulent Lawsonia intracellularis bacteria. Cell Numbers and Planting Information. A 300-ml stock suspension containing 4.54×10⁶ Sf9 cells per ml was used. Cells at four days of age were passed to 1000-ml spinner flasks. A total of 222 ml of fresh media was put into each spinner flask, and 1.25×10⁸ cells (27.5 ml of the stock suspension) were planted into the media, resulting in approximately 250 ml total volume with 0.5×10⁶ cells/ml.

Variable Description.

| Vessel Number | Temperature | Atmosphere | Seed (ml) |
|---|---|---|---|
| 1 | 27° C. | Specialty gas | 12.5 |
| 2 | 27° C. | CO₂ | 12.5 |
| 3 | 37° C. | Specialty gas | 12.5 |
| 4 | 37° C. | CO₂ | 12.5 |

Vessel Configuration. All vessels were configured with one fixed-length drop tube to 80% depth and a two-port SST assembly configured with 0.1 μm sterile filters.

Process Parameters. For Vessels 1 and 2, temperature was maintained at 27° C. For Vessels 3 and 4, temperature was maintained at 37° C. All vessels were agitated at 100 rpm. Oxygen (O₂) levels were variable. pH levels were not monitored or controlled. When establishing the specialty gas atmosphere in Vessels 1 and 3, the vessels were sparged with a specialty gas comprising 10% hydrogen, 10% CO₂ and 80% nitrogen that was filtered through a 0.1 μm filter to prevent contamination. The sparge rate was 5-10 cc/second for one minute for 250 ml of media. The sparge rate was 5-10 cc/second for two minutes for 500 ml of media. To prevent diffusion, vessels were hemostat closed after gassing. Vessels 2 and 4, which were maintained in a 5% CO₂ environment, possessed a 0.1 μm filter housing that was not hemostat closed. Hence, free gas exchange could occur with the 5% CO₂ environment via the filter housing.

Infection. The Sf9 cells were infected one day after they were planted in the vessels (Day 1). Seed culture was introduced into the vessels at a ratio of 1:20 of the vessel plant volume (i.e., 12.5 ml seed per 250 ml volume). Multiplicity of Infection (MOI) was not determined.

Media Supplementation. All vessels were supplemented with 250 ml of Ex-Cell™ 420 on Day 8 post planting of the Sf9 cells into the vessels.

Harvest. Samples were taken on Days 0, 1, 4, 7, 8 (pre-supplementation), 9, 10, 11, 14, 15, and 17 post-planting of the Sf9 cells into the vessels. On Day 11 post-planting, samples were obtained from Vessels 3 and 4. Because the cell viability and cell density were very low no further samples were taken and the remainder of the vessel contents was dispensed into large plastic vessels and frozen at minus 80° C. On Day 17 post-planting, samples were obtained from Vessels 1 and 2, and the remainder of the vessel contents was dispensed into large plastic vessels and frozen at minus 80° C.

Results. Sf9 cells grew better at 27° C. than at 37° C. (See Table 1). Lawsonia intracellularis grew in an environment of 27° C. and specialty gas (microaerophilic) conditions and under CO2 conditions. (microaerophilic was superior, however) (See Table 2).

TABLE 1
Viable Sf9 Cell Counts per Vessel*

| | Vessel 1 | Vessel 2 | Vessel 3 | Vessel 4 |
|---|---|---|---|---|
| | 27° C. + | 27° C. + | 37° C. + | 37° C.+ |
| | spec gas | CO₂ | spec gas | CO₂ |
| Day 0** | 5.0E+05 | 5.0E+05 | 5.0E+05 | 5.0E+05 |
| Day 1 | 7.4E+05 | 7.5E+05 | 3.6E+05 | 5.8E+05 |
| Day 4 | 2.0E+05 | 2.3E+06 | 1.40E+05 | 1.00E+05 |
| Day 7 | 1.7E+06 | 2.3E+06 | 2.48E+05 | 1.90E+05 |
| Day 8 presuppl. | 1.7E+06 | 2.3E+06 | 1.68E+05 | 1.48E+05 |
| Day 9 | 1.26E+06 | 1.19E+06 | 4.00E+04 | 3.40E+04 |
| Day 10 | 8.60E+05 | 1.65E+06 | 1.00E+04 | 1.20E+04 |
| Day 11 | 7.10E+05 | 9.00E+05 | 1.60E+04 | 1.00E+04 |
| Day 14 | 6.30E+05 | 1.07E+05 | na*** | na |
| Day 15 | **** | **** | na | na |
| Day 17 | **** | na | na | na |

*Data shown in scientific notation (e.g., 5.0E+05 = 5.0 × 10⁵)
**Number of days after planting of Sf9 cells into the vessels
***na = not analyzed

TABLE 2
L. intracellularis Copies per Vessel (qRT-PCR)*

| | Vessel 1 | Vessel 2 | Vessel 3 | Vessel 4 |
|---|---|---|---|---|
| | 27° C. + | 27° C. + | 37° C. + | 37° C.+ |
| | spec gas | CO₂ | spec gas | CO₂ |
| Day 1** | 7.00E+08 | 3.40E+08 | 1.30E+09 | 1.50E+09 |
| Day 4 | 9.00E+08 | 1.45E+09 | 1.25E+09 | 1.50E+09 |
| Day 7 | 2.90E+09 | 1.40E+09 | 1.60E+09 | 1.55E+09 |
| Day 8 presuppl | 2.65E+09 | 1.60E+09 | 1.35E+09 | 1.80E+09 |
| Day 8 postsuppl | 2.80E+09 | 1.50E+09 | 1.80E+09 | 1.90E+09 |
| Day 9 | 4.30E+09 | 1.00E+09 | 2.10E+09 | 2.20E+09 |
| Day 10 | 4.60E+09 | 1.30E+09 | 2.30E+09 | 2.40E+09 |
| Day 11 | 4.60E+09 | 1.50E+09 | 2.30E+09 | 2.10E+09 |
| Day 14 | 5.90E+09 | 2.20E+09 | na*** | na |
| Day 17 | 5.00E+09 | na | na | na |

*Data shown in scientific notation (e.g., 7.00E+08 = 7.00 × 10⁸)
**Time in hours post planting of Sf9 cells into the vessels
***na = not analyzed

### Example 2

PPE propagation experiment varying temperature, presence of serum, multiplicity of infection (MOI), and passage of Lawsonia intracellularis.

Purpose. The purpose of this experiment was to evaluate the growth of Lawsonia intracellularis using the Sf9 (spodoptera frugiperda) cell line at 27° centigrade (C.) versus 32° C. The purpose was also to evaluate the effect of the addition of 5% serum at 27° C. versus 32° C. It was also to evaluate the effect of increasing multiplicity of infection (MOI) at 27° C. versus 32° C. Finally, the purpose was to evaluate a second passage of Lawsonia intracellularis in Sf9 cells at 27° C.

Materials and Methods.

| | | Part (item | |
|---|---|---|---|
| | Description | number) | Lot |
| Parent cell | Sf9 | na¹ | Pass 8 |
| Growth media | Ex-Cell ™ 420 | 14420-1000 M | 5B0247 |
| Sera in the Growth Media | IFBS² | 12107-1000M | 3H0548 |
| Maintenance media (per | Ex-Cell ™ 420 | 14420-1000 M | 5B0247 |
| variable) | | | |
| Sera in the Maintenance | IFBS² | 12107-1000M | 3H0548 |
| media | | | |
| Live Lawsonia | Titer: | na¹ | na¹ |
| intracellularis | 5.0 dose/ml | | |

¹na = not applicable
²IFBS = Irradiated Fetal Bovine Serum

Cell and Media Information. The cell culture was Sf9 cells (Gibco® Cell Culture Systems, Invitrogen, Carlsbad, Calif., USA). The growth and maintenance media was Ex-Cell™ 420 Serum-Free Medium for Insect Cells with L-glutamine (JRH biosciences, Lenexa, Kans., USA; Catalog number 14420, item number 14420-1000M). The growth and maintenance media for vessels containing sera was Ex-Cell™ 420 Serum-Free Medium for Insect Cells with L-glutamine containing 5% Fetal Bovine Serum Sourced in USA gamma irradiated by SER-TAIN™ Process (JRH Biosciences, Lenexa, Kans., USA; Catalog number 12107, item number 12107-1000M). The seed culture contained modified live, non-virulent Lawsonia intracellularis bacteria.

Cell Numbers and Planting Information. A 300-ml stock suspension containing 5.20×10⁶ Sf9 cells per ml was used. Cells at three days of age were passed to 1000-ml spinner flasks. A total of 226 ml of fresh media was put into each spinner flask, and 1.25×10⁸ cells (24.0 ml of the stock suspension) were planted into the media, resulting in approximately 250 ml total volume with 0.5×10⁶ cells/ml.

Variable Description.

| Vessel Number | Temperature | 5% Serum | Seed (ml) |
|---|---|---|---|
| 1 | 27° C. | No | 6.25 |
| 2 | 32° C. | No | 6.25 |
| 3 | 27° C. | Yes | 6.25 |
| 4 | 32° C. | Yes | 6.25 |
| 5 | 27° C. | No | 22.00 |
| 6 | 32° C. | No | 22.00 |
| 7 | 27° C. | No | 35.7 from Example 1 |

Vessel Configuration. All vessels were configured with one fixed-length drop tube to 80% depth and a two-port SST assembly configured with 0.1 μm sterile filters.

Process Parameters. For Vessels 1, 3, 5, and 7, temperature was maintained at 27° C. For Vessels 2, 4, and 6, temperature was maintained at 32° C. All vessels were agitated at 100 rpm. Oxygen (O₂) levels were variable. pH levels were not monitored or controlled. The atmosphere above the media in all vessels was the specialty gas. When establishing the atmosphere in the vessels, the vessels were sparged with a specialty gas comprising 10% hydrogen, 10% CO₂ and 80% nitrogen that was filtered through a 0.1 μm filter to prevent contamination. The sparge rate was 5-10 cc/second for one minute for 250 ml of media. The sparge rate was 5-10 cc/second for two minutes for 500 ml of media. To prevent diffusion, vessels were hemostat closed after gassing.

Infection. The Sf9 cells were infected when they were planted in the vessels (Day 0). Seed culture was introduced into Vessels 1, 2, 3, and 4 at a ratio of 1:40 of the vessel plant volume (i.e., 6.25 ml seed per 250 ml volume). Seed culture was introduced into Vessels 5 and 6 at a ratio of approximately 1:11.4 of the vessel plant volume (i.e., 22 ml seed per 250 ml volume). Seed culture was not introduced into Vessel 7. Rather, 35.7 ml of the sample harvested on Day 17 post-planting from Vessel 1 of Example 1 above was introduced into Vessel 7 (a ratio of 1:7 of inoculum to vessel plant volume). Multiplicity of Infection (MOI) was not determined.

Media Supplementation. All vessels were supplemented with 250 ml of Ex-Cell™ 420 or 250 ml of Ex-Cell™ 420 plus fetal bovine sera, as appropriate, on Day 6 post planting of the Sf9 cells into the vessels.

Harvest. Samples were taken on Days 0, 1, 4, 6 (presupplementation), 8, 11, 13, 15, 18, and 20 post-planting of the Sf9 cells into the vessels. After obtaining the Day 20 samples from Vessels 2, 4, 6, and 7, the remainder of the vessel contents was dispensed into large plastic vessels and frozen at minus 80° C. Samples were taken from Vessels 1, 3, and 5 (which were maintained at 27° C.) on Day 25, and the remainder of the vessel contents was dispensed into large plastic vessels and frozen at minus 80° C.

Results. Sf9 cells grew better at 27° C. than at 32° C. (See Table 3). As seen in Table 4, the Lawsonia grew in every condition except for Vessel 7, which was inoculated with inoculum from Example 1 (i.e., the 2^{nd} passage). This is likely due to a non-viable inoculum from Example 1. In general, the Lawsonia achieved higher levels of growth when grown at 27° C. and without serum. Although the highest Lawsonia copies per ml were observed when using a high MOI, there appears to be a diminishing return (i.e., a 100 fold return of investment was seen at a lower MOI compared with a 46 fold return at a higher MOI—See Table 5). Lawsonia grew when maintained at 32° C.; however, these infections were characterized as producing Lawsonia quickly, but not maintaining strong growth.

TABLE 3
Viable Sf9 Cell Counts per Vessel*

| | | | Vessel 3 | Vessel 4 | Vessel 5 | Vessel 6 | Vessel 7 |
|---|---|---|---|---|---|---|---|
| | Vessel 1 | Vessel 2 | 27° C. + | 32° C. + | 27° C. + Hi | 32° C. + Hi | 27° C. + Ex 1 |
| | 27° C. | 32° C. | FBS | FBS | MOI | MOI | Lawsonia |
| Day 0** | 5.0E+05 | 5.0E+05 | 5.0E+05 | 5.0E+05 | 5.0E+05 | 5.0E+05 | 5.0E+05 |
| Day 1 | 4.0E+05 | 6.3E+05 | 4.5E+05 | 3.9E+05 | 3.9E+05 | 6.0E+05 | 3.0E+05 |
| Day 4 | 1.3E+06 | 6.2E+05 | 1.23E+06 | 9.70E+05 | 1.92E+06 | 4.30E+05 | 1.16E+06 |
| Day 6 presuppl | 2.4E+06 | 5.5E+05 | 1.65E+06 | 8.50E+05 | 1.18E+06 | 6.50E+05 | 1.32E+06 |
| Day 8 | 1.19E+06 | 2.28E+05 | 6.20E+05 | 4.18E+05 | 7.50E+05 | 3.60E+05 | 1.75E+06 |
| Day 11 | 9.00E+05 | 3.14E+05 | 5.00E+05 | 3.76E+05 | 9.30E+05 | 3.18E+05 | 1.25E+06 |
| Day 13 | 5.80E+05 | 2.20E+05 | 4.80E+05 | 2.74E+05 | 5.30E+05 | 2.00E+05 | 1.05E+06 |
| Day 15 | 7.70E+05 | 1.58E+05 | 5.80E+05 | 3.10E+05 | 5.40E+05 | 2.10E+05 | 1.00E+06 |
| Day 18 | 5.10E+05 | 1.48E+05 | 5.10E+05 | 1.90E+05 | 5.80E+05 | 1.50E+05 | 6.10E+05 |
| Day 20 | 4.60E+05 | 1.16E+05 | 4.40E+05 | 1.90E+05 | 5.70E+05 | 1.60E+05 | 5.70E+05 |
| Day 25 | 3.00E+05 | na*** | 3.80E+05 | na | 4.30E+05 | na | na |

*Data shown in scientific notation (e.g., 5.0E+05 = 5.0 × 10⁵)
**Number of days after planting of Sf9 cells into the vessels
***na = not analyzed

TABLE 4
L. intracellularis Copies per Vessel (qRT-PCR)*

| | | | Vessel 3 | Vessel 4 | Vessel 5 | Vessel 6 | Vessel 7 |
|---|---|---|---|---|---|---|---|
| | Vessel 1 | Vessel 2 | 27° C. + | 32° C. + | 27° C. + Hi | 32° C. + Hi | 27° C. + Ex 1 |
| | 27° C. | 32° C. | FBS | FBS | MOI | MOI | Lawsonia |
| Day 0** | 1.30E+09 | 1.40E+09 | 9.50E+08 | 4.50E+08 | 6.50E+09 | 6.50E+09 | 5.00E+08 |
| Day 4 | 1.50E+09 | 3.45E+09 | 1.65E+09 | 5.00E+09 | 3.35E+09 | 2.15E+10 | 4.75E+08 |
| Day 6 presuppl | 1.80E+09 | 2.25E+10 | 2.70E+09 | 6.00E+09 | 4.90E+09 | 2.65E+10 | 4.05E+08 |
| Day 6 postsuppl | 1.50E+09 | 1.60E+10 | 2.40E+09 | 3.40E+09 | 4.00E+09 | 2.50E+10 | 3.20E+08 |
| Day 8 | 2.40E+09 | 3.00E+10 | 2.60E+09 | 6.80E+09 | 1.10E+10 | 4.60E+10 | 4.50E+08 |
| Day 11 | 2.00E+10 | 7.50E+10 | 8.40E+09 | 9.60E+09 | 6.90E+10 | 3.70E+10 | 3.20E+08 |
| Day 13 | 3.90E+10 | 7.90E+10 | 9.90E+09 | 6.70E+09 | 1.20E+11 | 2.90E+10 | 3.50E+08 |
| Day 15 | 4.00E+10 | 5.60E+10 | 8.20E+09 | 5.10E+09 | 1.10E+11 | 2.40E+10 | 3.10E+08 |
| Day 17 | 5.10E+10 | 6.40E+10 | 1.30E+10 | 3.80E+09 | 1.80E+11 | 1.90E+10 | 3.20E+08 |
| Day 20 | 8.90E+10 | 5.50E+10 | 1.50E+10 | 3.80E+09 | 1.70E+11 | 1.70E+10 | 2.80E+08 |
| Day 22 | 1.10E+11 | na*** | 1.60E+10 | na | 1.70E+11 | na | na |
| Day 25 | 1.30E+11 | na | 1.20E+10 | na | 2.40E+11 | na | na |
| Day 27 | 1.20E+11 | na | 1.30E+10 | na | 3.00E+11 | na | na |

*Data shown in scientific notation (e.g., 1.30E+09 = 1.30 × 10⁹)
**Number of days after planting of Sf9 cells into the vessels
***na = not analyzed

TABLE 5
Fold increase in L. intracellularis

| | | | Vessel 3 | Vessel 4 | Vessel 5 | Vessel 6 | Vessel 3 |
|---|---|---|---|---|---|---|---|
| | Vessel 1 | Vessel 2 | 27° C. + | 32° C. + | 27° C. + Hi | 32° C. + Hi | 27° C. + Ex 1 |
| | 27° C. | 32° C. | FBS | FBS | MOI | MOI | Lawsonia |
| Increase Day 0-6 | 1.4 | 16.1 | 2.8 | 13.3 | 0.8 | 4.1 | 0.8 |
| Increase Day 6-13 | 21.7 | 3.5 | 3.7 | 1.1 | 24.5 | 1.1 | 0.9 |
| Increase Day 13-20 | 2.3 | 0.7 | 1.5 | 0.6 | 1.4 | 0.6 | 0.8 |
| Increase Day 0-13 | 30.0 | 56.4 | 10.4 | 14.9 | 18.5 | 4.5 | 0.7 |
| Increase Day 0-20 | 68.5 | 39.3 | 15.8 | 8.4 | 26.2 | 2.6 | 0.6 |
| Maximum increase | 100.0 | 56.4 | 16.8 | 21.3 | 46.2 | 7.1 | na* |

*na = not applicable

### Example 3

PPE propagation experiment varying temperature, multiplicity of infection (MOD, and supplementation with media, assessing temperature adaptation, and generating Sf9 bacterial seed at various harvest time points.

Purpose. The purpose of this experiment was to evaluate the growth of Lawsonia intracellularis using the Sf9 (spodoptera frugiperda) cell line at 27° centigrade (C.), 29.5° C., and 32° C. The purpose was also to evaluate effect of varying multiplicity of infection (MOI) at 27° C. It was also to evaluate the repeated supplementation on at various time points at 27° C. The purpose also included the evaluation of temperature adaptation of Sf9 cells from 27° C. to 29.5° C. and then to 32° C. It also included the evaluation of the growth of Lawsonia intracellularis during Days 0-6 at 32° C. followed by growth during Days 6-completion at 29.5° C. Finally, the purpose was to generate Lawsonia intracellularis bacterial seed at various harvest time points for later inoculation to confirm passage feasibility.

Materials and Methods.

| | | Part (item | |
|---|---|---|---|
| | Description | number) | Lot |
| Parent cell | Sf9 | na¹ | Pass 7 |
| Growth media | Ex-Cell ™ 420 | 14420-1000M | 5C0415 |
| Maintenance media (per | Ex-Cell ™ 420 | 14420-1000 M | 5C0416 |
| variable) | | | |
| Live Lawsonia | Titer: | na¹ | na¹ |
| intracellularis | 5.0 dose/ml | | |

¹na = not applicable

Cell and Media Information. The cell culture was Sf9 cells (Gibco® Cell Culture Systems, Invitrogen, Carlsbad, Calif., USA). The growth and maintenance media was Ex-Cell™ 420 Serum-Free Medium for Insect Cells with L-glutamine (JRH biosciences, Lenexa, Kans., USA; Catalog number 14420, item number 14420-1000M). The seed culture contained modified live, non-virulent Lawsonia intracellularis bacteria.

Cell Numbers and Planting Information. Three stock solutions were used. The first was a 300-ml stock suspension maintained at 27° C. containing 3.99×10⁶ Sf9 cells per ml (viability of 87.3%). The second was a 300-ml stock suspension maintained at 29.5° C. containing 1.96×10⁶ Sf9 cells per ml (viability of 77.6%). The third was a 300-ml stock suspension maintained at 32° C. containing 0.9×10⁶ Sf9 cells per ml (viability of 52.8%). Cells at three days of age were passed to 1000-ml spinner flasks. A total of 219 ml of fresh media was put into spinner flask numbers 1-4, and 1.25×10⁸ cells (31.0 ml of the 27° C. stock suspension) were planted into the media, resulting in 250 ml total volume with 0.5×10⁶ cells/ml. A total of 186 ml of fresh media was put into spinner flask numbers 5 and 6, and 1.25×10⁸ cells (64.0 ml of the 29.5° C. stock suspension) were planted into the media, resulting in 250 ml total volume with 0.5×10⁶ cells/ml. A total of 112 ml of fresh media was put into spinner flask number 7, and 1.25×10⁸ cells (138.0 ml of the 32° C. stock suspension) were planted into the media, resulting in 250 ml total volume with 0.5×10⁶ cells/ml.

| Vessel | Growth | | Parent | Seed | | Media |
|---|---|---|---|---|---|---|
| Number | Temperature | | Temperature | (ml) | Doses | Supplement |
| 1 | 27° | C. | 27° C. | 6.25 | 31.25 | Day 6 |
| 2 | 27° | C. | 27° C. | 1.56 | 7.80 | Day 6 |
| 3 | 27° | C. | 27° C. | 0.40 | 2.00 | Day 6 |
| 4 | 27° | C. | 27° C. | 6.25 | 31.25 | Days 6, 13, 19 |
| 5 | 29.5° | C. | 29.5° C. | 6.25 | 31.25 | Day 6 |
| 6 | 32 then | | 29.5° C. | 6.25 | 31.25 | Day 6 |
| | 29.5° C. | | | | | |
| 7 | 32° | C. | 32° C. | 6.25 | 31.25 | Day 6 |

Vessel Configuration. All vessels were configured with one fixed-length drop tube to 80% depth and a two-port SST assembly configured with 0.1 μm sterile filters.

Process Parameters. For Vessels 1, 2, 3, and 4, temperature was maintained at 27° C. For Vessel 5, temperature was maintained at 29.5° C. For Vessel 6, the temperature of the parent media was 29.5° C. It was raised to 32° C. on Day 0 and maintained at this temperature for Days 0 to 6, then decreased to 29.5° C. for Days 6 to 24. For Vessel 7, temperature was maintained at 32° C. Vessels were agitated at 100 rpm. Oxygen (O₂) levels were variable. pH levels were not monitored or controlled. The atmosphere above the media in all vessels was the specialty gas. When establishing the atmosphere in the vessels, the vessels were sparged with a specialty gas comprising 10% hydrogen, 10% CO₂ and 80% nitrogen that was filtered through a 0.1 μm filter to prevent contamination. The sparge rate was 5-10 cc/second for one minute for 250 ml of media. The sparge rate was 5-10 cc/second for two minutes for 500 ml of media. To prevent diffusion, vessels were hemostat closed after gassing.

Infection. The Sf9 cells were infected when they were planted in the vessels (Day 0). Seed culture was introduced into Vessels 1, 4, 5, 6, and 7 at a ratio of 1:40 of the vessel plant volume (i.e., 6.25 ml seed per 250 ml volume). Seed culture was introduced into Vessel 2 at a ratio of approximately 1:160 of the vessel plant volume (i.e., 1.56 ml seed per 250 ml volume). Seed culture was introduced into Vessel 3 at a ratio of approximately 1:640 of the vessel plant volume (i.e., 0.4 ml seed per 250 ml volume). Multiplicity of Infection (MOI) was not determined.

Media Supplementation. Vessels 1, 2, 3, 4, 5, 6, and 7 were supplemented with 250 ml of Ex-Cell™ 420 on Day 6 post planting of the Sf9 cells into the vessels. For Vessel 4, on Days 13 and 19 post planting of the Sf9 cells into the vessel, 250 ml of the cell culture was transferred to an empty 1000 ml vessel and supplemented with an additional 250 ml of Ex-Cell™ 420.

Harvest. Samples were taken on Days 0, 3, 5, 6 (presupplementation), 7, 10, 13, 17, 19, 20, 24, and 27 post planting of the Sf9 cells into the vessels. However, samples were not taken from Vessel 7 on Days 20, 24, and 27. For Vessel 4, 25 ml of media and cells were harvested and frozen on Days 6, 13, 19, and 24 (prior to supplementation of media on Days 6, 13, and 19). After obtaining the Day 19 sample from Vessel 7 and the Day 27 samples from Vessels 1-6, the remainder of the vessel contents was dispensed into large plastic vessels and frozen at minus 80° C.

Results. The conditions for Vessel 1 and Vessel 4 were similar except that Vessel 4 was additionally supplemented on Days 13 and 19. This additional supplementation resulted in healthier Sf9 cells as determined by cell density and viability (See Tables 6 and 7), and a higher overall increase in the yield of Lawsonia (See Table 8). Increases in the yield of Lawsonia were realized in Vessels 1-4, which were maintained at 27° C.

The conditions for Vessels 1, 5, 6, and 7 were similar except for the temperature of the temperature of the parent cells and/or temperature during the growth of Lawsonia. The Lawsonia propagation was accelerated during the early infection period (Days 0-6) when conducted at 32° C. (Vessel 6). This was not duplicated in Vessel 7, most likely due to poor Sf9 viability (50%) at the time of infection. Thus, although propagation of Lawsonia at 27° C. took longer to achieve maximum growth, a greater total yield was realized.

TABLE 6
Viable Sf9 Cell Counts per Vessel*

| | | | | Vessel 4 | | | |
|---|---|---|---|---|---|---|---|
| | Vessel 1 | Vessel 2 | Vessel 3 | 27° C. + 31 | Vessel 5 | Vessel 6 | Vessel 7 |
| | 27° C. + | 27° C. + | 27° C. + | doses + | 29.5° C. + | 32°/29.5° C. + | 32° C. + |
| | 31 doses | 8 doses | 2 doses | Suppl's | 31 doses | 31 doses | 31 doses |
| Day 0** | 5.0E+05 | 5.0E+05 | 5.0E+05 | 5.0E+05 | 5.0E+05 | 5.0E+05 | 5.0E+05 |
| Day 3 | 2.4E+06 | 3.0E+06 | 2.10E+06 | 1.76E+06 | 2.04E+06 | 9.40E+05 | 4.80E+05 |
| Day 5 | 2.2E+06 | 3.7E+06 | 3.04E+06 | 2.50E+06 | 2.00E+06 | 1.14E+06 | 4.40E+05 |
| Day 6 presuppl | 2.1E+06 | 2.8E+06 | 2.24E+06 | 1.80E+06 | 2.10E+06 | 1.10E+06 | 7.80E+05 |
| Day 7 | 1.2E+06 | 1.4E+06 | 1.27E+06 | 9.70E+05 | 9.60E+05 | 7.00E+05 | 2.52E+05 |
| Day 10 | 8.50E+05 | 1.31E+06 | 1.39E+06 | 2.10E+06 | 9.00E+05 | 6.10E+05 | 1.40E+05 |
| Day 13 | 8.00E+05 | 1.61E+06 | 1.04E+06 | 2.33E+06 | 5.20E+05 | 3.00E+05 | 1.10E+05 |
| Day 17 | 6.30E+05 | 1.13E+06 | 5.50E+05 | 1.06E+06 | 6.50E+05 | 3.00E+05 | 9.00E+04 |
| Day 19 | 4.30E+05 | 7.00E+05 | 5.90E+05 | 1.03E+06 | 3.70E+05 | 2.60E+05 | 5.00E+04 |
| Day 20 | 4.50E+05 | 7.40E+05 | 4.20E+05 | 4.40E+05 | 6.20E+05 | 4.20E+05 | na*** |
| Day 24 | 4.50E+05 | 4.80E+05 | 3.20E+05 | 3.80E+05 | 3.20E+05 | 4.00E+05 | na |

*Data shown in scientific notation (e.g., 5.0E+05 = 5.0 × 10⁵)
**Number of days after planting of Sf9 cells into the vessels
***na = not analyzed

TABLE 7
Sf9 Cell Viability by Vessel (Percent)

| | | | | Vessel 4 | | | |
|---|---|---|---|---|---|---|---|
| | Vessel 1 | Vessel 2 | Vessel 3 | 27° C. + | Vessel 5 | Vessel 6 | Vessel 7 |
| | 27° C. + | 27° C. + | 27° C. + | 31 doses + | 29.5° C. + | 32°/29.5° C. + | 32° C. + |
| | 31 doses | 8 doses | 2 doses | Suppl's | 31 doses | 31 doses | 31 doses |
| Day 0* | 87.3 | 87.3 | 87.3 | 87.3 | 77.6 | 77.6 | 52.8 |
| Day 3 | 99.6 | 99.7 | 99.5 | 100.0 | 99.0 | 94.9 | 53.3 |
| Day 5 | 98.2 | 97.4 | 97.8 | 98.4 | 88.9 | 82.6 | 44.0 |
| Day 6 presuppl | 97.2 | 97.9 | 99.1 | 98.4 | 89.4 | 71.4 | 50.8 |
| Day 7 | 92.2 | 94.5 | 97.7 | 94.2 | 76.2 | 72.9 | 38.7 |
| Day 10 | 85.0 | 85.6 | 91.4 | 99.1 | 64.3 | 55.0 | 25.5 |
| Day 13 | 76.2 | 72.9 | 83.2 | 98.3 | 50.0 | 29.4 | 14.1 |
| Day 17 | 57.3 | 52.1 | 52.9 | 88.3 | 33.8 | 19.6 | 11.4 |
| Day 19 | 55.8 | 44.0 | 50.0 | 87.3 | 26.2 | 18.6 | 5.2 |
| Day 20 | 44.1 | 39.6 | 33.1 | 83.0 | 22.0 | 20.8 | na** |
| Day 24 | 37.5 | 26.4 | 28.8 | 65.5 | 13.7 | 26.7 | na |

*Number of days after planting of Sf9 cells into the vessels
**na = not analyzed

TABLE 8
L. intracellularis Copies per Vessel (qRT-PCR)*

| | | | | Vessel 4 | | | |
|---|---|---|---|---|---|---|---|
| | Vessel 1 | Vessel 2 | Vessel 3 | 27° C. + 31 | Vessel 5 | Vessel 6 | Vessel 7 |
| | 27° C. + | 27° C. + | 27° C. + | doses + | 29.5° C. + | 32°/29.5° C. + | 32° C. + |
| | 31 doses | 8 doses | 2 doses | Suppl's | 31 doses | 31 doses | 31 doses |
| Day 0** | 1.20E+09 | 9.00E+08 | 1.00E+08 | 1.55E+09 | 1.60E+09 | 1.60E+09 | 1.25E+09 |
| Day 4 | 1.30E+09 | 3.25E+08 | 9.50E+07 | 1.65E+09 | 1.95E+09 | 4.15E+09 | 2.30E+09 |
| Day 5 | 2.50E+09 | 5.00E+08 | 1.80E+08 | 4.80E+09 | 2.60E+09 | 1.80E+10 | 3.35E+09 |
| Day 6 presuppl | 2.20E+09 | 1.30E+09 | 1.80E+08 | 3.15E+09 | 2.50E+09 | 1.90E+10 | 3.85E+09 |
| Day 6 postsuppl | 2.80E+09 | 1.10E+09 | 2.20E+08 | 4.50E+09 | 2.70E+09 | 1.40E+10 | 3.70E+09 |
| Day 7 | 3.00E+09 | 1.30E+09 | 3.60E+08 | 4.50E+09 | 3.20E+09 | 1.00E+10 | 3.50E+09 |
| Day 10 | 1.50E+10 | 1.70E+09 | 6.90E+08 | 1.70E+10 | 8.20E+09 | 5.40E+10 | 5.50E+09 |
| Day 13 presuppl | 5.20E+10 | 1.50E+09 | 1.70E+09 | 4.80E+10 | 1.30E+10 | 5.60E+10 | 3.40E+09 |
| Day 13 postsuppl | na*** | na | na | 4.60E+10 | na | na | na |
| Day 17 | 2.10E+10 | 2.80E+09 | 1.10E+09 | 3.20E+10 | 2.30E+10 | 4.00E+10 | 3.20E+09 |
| Day 19 presuppl | 3.30E+10 | 1.70E+09 | 7.20E+08 | 5.00E+10 | 3.40E+10 | 2.40E+10 | 3.30E+09 |
| Day 19 postsuppl | na | na | na | 3.12E+10 | na | na | na |
| Day 24 | 6.80E+10 | 7.80E+08 | 1.40E+09 | 6.00E+10 | 4.40E+10 | 5.20E+10 | na |
| Day 27 | 9.60E+10 | 2.30E+08 | 7.80E+08 | 1.56E+11 | 4.30E+10 | 8.80E+10 | na |

*Data shown in scientific notation (e.g., 1.20E+09 = 1.20 × 10⁹)
**Number of days after planting of Sf9 cells into the vessels
***na = not analyzed

TABLE 9
Fold increase (qRT-PCR) in Lawsonia

| | | | | Vessel 4 | | | |
|---|---|---|---|---|---|---|---|
| | Vessel 1 | Vessel 2 | Vessel 3 | 27° C. + 31 | Vessel 5 | Vessel 6 | Vessel 7 |
| | 27° C. + | 27° C. + | 27° C. + | doses + | 29.5° C. + | 32°/29.5° C. + | 32° C. + |
| | 31 doses | 8 doses | 2 doses | Suppl's | 31 doses | 31 doses | 31 doses |
| Increase Day 0-6* | 1.8 | 1.4 | 1.8 | 2.0 | 1.6 | 11.9 | 3.1 |
| Increase Day 6-13 | 23.6 | 1.2 | 9.4 | 15.2 | 5.2 | 2.9 | 0.9 |
| Increase Day 13-19 | 0.6 | 1.1 | 0.4 | 1.0 | 2.6 | 0.4 | 1.0 |
| Increase Day 19-27 | 2.9 | 0.1 | 1.1 | 3.1 | 1.3 | 3.7 | na** |
| Increase Day 0-13 | 43.3 | 1.7 | 17.0 | 31.0 | 8.1 | 35.0 | 2.7 |
| Increase Day 0-19 | 27.5 | 1.9 | 7.2 | 32.3 | 21.3 | 15.0 | 2.6 |
| Increase Day 0-27 | 80.0 | 0.3 | 7.8 | 100.6 | 26.9 | 55.0 | na |
| Maximum increase | 80.0 | 3.1 | 17.0 | 100.6 | 27.5 | 55.0 | 4.4 |

*Number of days after planting of Sf9 cells into the vessels
**na = not analyzed

### Example 4

PPE propagation experiment evaluating Lawsonia intracellularis bacteria growth for samples from varying harvest dates, and Lawsonia intracellularis temperature adaptation.

Purpose. The purpose of this experiment was to evaluate the growth of Lawsonia intracellularis bacteria that were harvested at four different time points post infection of the Sf9 (spodoptera frugiperda) cell line at 27° centigrade (C.) and to ensure that Lawsonia propagated in Sf9 cells can reinfect new cultures of Sf9 cells. The purpose was also to evaluate the growth of Lawsonia intracellularis bacteria during Days 0-6 at 32° C. followed by growth during Days 6-completion at 27° C.

Materials and Methods.

| | | Part (item | |
|---|---|---|---|
| | Description | number) | Lot |
| Parent cell | Sf9 | na¹ | Pass 13 |
| Growth media | Ex-Cell ™ 420 | 14420-1000M | 5C0416 |
| Maintenance media (per variable) | Ex-Cell ™ 420 | 14420-1000M | 5C0416 |
| L. intracellularis pass 1, vessel 4, Ex 3 | 1.28 × 10⁷ copies/ml | na¹ | Day 6 |
| L. intracellularis pass 1, vessel 4, Ex 3 | 9.6 × 10⁷ copies/ml | na¹ | Day 13 |
| L. intracellularis pass 1, vessel 4, Ex 3 | 5.0 × 10⁷ copies/ml | na¹ | Day 19 |
| L. intracellularis pass 1, vessel 4, Ex 3 | ~2.6 × 10⁷ copies/ml | na¹ | Day 24 |

¹na = not applicable

Cell and Media Information. The cell culture was Sf9 cells (Gibco® Cell Culture Systems, Invitrogen, Carlsbad, Calif., USA). The growth and maintenance media was Ex-Cell™ 420 Serum-Free Medium for Insect Cells with L-glutamine (JRH biosciences, Lenexa, Kans., USA; Catalog number 14420, item number 14420-1000M). The seed culture containing modified live, non-virulent Lawsonia intracellularis bacteria was obtained during Example 3. As described above, 25 ml samples were harvested from Vessel 4 on Days 6, 13, 19, and 24 and frozen at −80° C. Cell Numbers and Planting Information. A 300-ml stock suspension maintained in a 1000 L spinner flask at 27° C. was used. At Day 6 post planting the vessel contained 6.4×10⁶ Sf9 cells per ml (viability of 99.7%) in Ex-Cell™ 420 media. Cells at six days of age were passed to five new 500-ml spinner flasks. A total of 230.5 ml of fresh media was put into each spinner flask, and 1.25×10⁸ cells (19.5 ml of the stock suspension) were planted into the media, resulting in 250 ml total volume with 0.5×10⁶ cells/ml.

Variable Description.

| Vessel | Growth | | Harvest Day | Volume of | Media |
|---|---|---|---|---|---|
| Number | Temperature | | of Seed | Seed (ml) | Supplement |
| 1 | 27° | C. | 6 | 25.0 | Day 6 |
| 2 | 27° | C. | 13 | 3.3 | Day 6 |
| 3 | 27° | C. | 19 | 6.3 | Day 6 |
| 4 | 27° | C. | 24 | 12.1 | Day 6 |
| 5 | 32 then 27° | C. | 13 | 3.3 | Day 6 |

Vessel Configuration. All vessels were configured with one fixed-length drop tube to 80% depth and a two-port SST assembly configured with 0.1 μm sterile filters.

Process Parameters. For Vessels 1, 2, 3, and 4, temperature was maintained at 27° C. For Vessel 5, the temperature was maintained at 32° C. on Days 0 to 6, then decreased to 27° C. for Days 6 to 29. All vessels were agitated at 100 rpm. Oxygen (O₂) levels were variable. pH levels were not monitored or controlled. The atmosphere above the media in all vessels was the specialty gas. When establishing the atmosphere in the vessels, the vessels were sparged with a specialty gas comprising 10% hydrogen, 10% CO₂ and 80% nitrogen that was filtered through a 0.1 μm filter to prevent contamination. The sparge rate was 5-10 cc/second for one minute for 250 ml of media. The sparge rate was 5-10 cc/second for two minutes for 500 ml of media. To prevent diffusion, vessels were hemostat closed after gassing. Infection. For all vessels, the Sf9 cells were infected when they were planted in the vessels (Day 0). At the time of infection, multiplicity of infection (MOI) by Lawsonia intracellularis bacteria was calculated using the qRT-PCR results from Example 3.

qRT-PCR Results of L. intracellularis seed Vessel 4, Example 3

| Day 6 seed | Day 13 seed | Day 19 seed | Day 24 seed |
|---|---|---|---|
| 1.26 × 10⁷ copies/ml | 9.6 × 10⁷ copies/ml | 5.0 × 10⁷ copies/ml | ~2.6 × 10⁷ copies/ml |

The target infection amount was 3.15×10⁸ copies of L. intracellularis per vessel. Vessel 1 was infected with 25.0 ml of the Day 6 seed (3.15×10⁸ copies/1.26×10⁷ copies/ml). Vessels 2 and 5 were infected with 3.3 ml of the Day 13 seed (3.15×10⁸ copies/9.6×10⁷ copies/ml). Vessel 3 was infected with 6.3 ml of the Day 19 seed (3.15×10⁸ copies/5.0×10⁷ copies/ml). Vessel 4 was infected with 12.1 ml of the Day 24 seed (3.15×10⁸ copies/2.6×10⁷ copies/ml).

Media Supplementation. On Day 6 post planting of the Sf9 cells into the vessels, all vessels were supplemented with 250 ml of Ex-Cell™ 420.

Harvest. Samples were taken on Days 0, 3, 6 (presupplementation), 8, 10, 14, 17, 21, 25, and 29 post planting of the Sf9 cells into the vessels. However, a sample was not taken from Vessel 5 on Day 29 because the cell count was low. After obtaining the Day 25 sample from Vessel 5 and the Day 29 samples from Vessels 1-4, the remainder of the vessel contents was dispensed into large plastic vessels and frozen at minus 80° C.

Results. The results demonstrate that Lawsonia previously passaged in insect cells can be harvested and used to infect new insect cells (See Tables 10, 11, and 12). The 67-fold increase of the Day 24 seed is comparable to the 80-100 fold yields that had been observed for fresh seed used in earlier examples. An early Lawsonia propagation burst was observed in the cultures begun at 32° C. and switched to 27° C., but these cultures did not sustain the level of growth observed in the cultures maintained at 27° C. for the entire experiment. Finally, it is unclear why the standardized infections of the Day 6, 13, 19 and 24 seeds did not achieve “identical” yield increases.

TABLE 10
Viable Sf9 Cell Counts per Vessel*

| | Vessel 1. | Vessel 2. | Vessel 3. | Vessel 4. | Vessel 5. |
|---|---|---|---|---|---|
| | 27° C. + | 27° C. + | 27° C. + | 27° C. + | 32/27° C. + |
| | Day 6 | Day 13 | Day 19 | Day 24 | Day 13 |
| | Lawsonia | Lawsonia | Lawsonia | Lawsonia | Lawsonia |
| Day 0** | 5.0E+05 | 5.0E+05 | 5.0E+05 | 5.0E+05 | 5.0E+05 |
| Day 3 | 2.6E+06 | 2.2E+06 | 2.58E+06 | 2.02E+06 | 1.44E+06 |
| Day 6 | 3.6E+06 | 2.8E+06 | 2.48E+06 | 2.20E+06 | 6.40E+05 |
| presuppl | | | | | |
| Day 8 | 1.7E+06 | 1.5E+06 | 1.49E+06 | 1.13E+06 | 2.10E+05 |
| Day 10 | 2.19E+06 | 1.87E+06 | 1.39E+06 | 1.26E+06 | 9.00E+04 |
| Day 14 | 1.51E+06 | 1.59E+06 | 8.70E+05 | 4.00E+05 | 9.00E+04 |
| Day 17 | 1.29E+06 | 9.50E+05 | 1.05E+06 | 8.10E+05 | 3.00E+04 |
| Day 21 | 1.08E+06 | 7.40E+05 | 6.10E+05 | 4.90E+05 | 1.00E+04 |
| Day 25 | **** | **** | **** | **** | **** |
| Day 29 | **** | **** | **** | **** | na*** |

*Data shown in scientific notation (e.g., 5.0E+05 = 5.0 × 10⁵)
**Number of days after planting of Sf9 cells into the vessels
***na = not analyzed

TABLE 11
L. intracellularis Copies per Vessel (qRT-PCR)*

| | Vessel 1. | Vessel 2. | Vessel 3. | Vessel 4. | Vessel 5. |
|---|---|---|---|---|---|
| | 27° C. + | 27° C. + | 27° C. + | 27° C. + | 32/27° C. + |
| | Day 6 | Day 13 | Day 19 | Day 24 | Day 13 |
| | Lawsonia | Lawsonia | Lawsonia | Lawsonia | Lawsonia |
| Day 0** | 6.50E+07 | 2.25E+07 | 1.90E+08 | 1.00E+08 | 3.65E+07 |
| Day 6 | 6.60E+07 | 1.20E+08 | 3.20E+08 | 7.00E+08 | 4.20E+08 |
| post suppl | | | | | |
| Day 10 | 2.00E+08 | 2.00E+08 | 4.80E+08 | 3.40E+09 | 3.70E+08 |
| Day 14 | 2.00E+08 | 4.50E+08 | 6.80E+08 | 2.30E+09 | 3.00E+08 |
| Day 17 | 3.00E+08 | 6.10E+08 | 6.80E+08 | 6.20E+09 | 1.70E+08 |
| Day 25 | 9.00E+08 | 6.90E+08 | 7.40E+08 | 5.60E+09 | 3.00E+08 |
| Day 29 | 5.90E+08 | 5.80E+08 | 6.70E+08 | 6.70E+09 | 3.50E+08 |

*Data shown in scientific notation (e.g., 6.50E+07 = 6.50 × 10⁷)
**Number of days after planting of Sf9 cells into the vessels

TABLE 12
Fold increase (qRT-PCR) in L. intracellularis

| | Vessel 1. | Vessel 2. | Vessel 3. | Vessel 4. | Vessel 5. |
|---|---|---|---|---|---|
| | 27° C. + | 27° C. + | 27° C. + | 27° C. + | 32/27° C. + |
| | Day 6 | Day 13 | Day 19 | Day 24 | Day 13 |
| | Lawsonia | Lawsonia | Lawsonia | Lawsonia | Lawsonia |
| Increase | 1.0 | 5.3 | 1.7 | 7.0 | 11.5 |
| Day 0-6* | | | | | |
| Increase | 3.0 | 3.8 | 2.1 | 3.3 | 0.7 |
| Day 6-14 | | | | | |
| Increase | 4.5 | 1.5 | 1.1 | 2.4 | 1.0 |
| Day 14-25 | | | | | |
| Increase | 3.1 | 8.9 | 2.5 | 34.0 | 10.1 |
| Day 0-10 | | | | | |
| Maximum | 13.8 | 30.7 | 3.9 | 67.0 | 11.5 |
| Increase | | | | | |

*Number of days after planting of Sf9 cells into the vessels

### Example 5

PPE propagation experiment comparing types of media, multiplicity of infection (MOI), and infected versus uninfected Sf9 cells for analysis.

Purpose. The purpose of this experiment was to compare the growth of Lawsonia intracellularis using the Sf9 (spodoptera frugiperda) cell line in either Ex-Cell™ 420 media or IPL-41 media. The purpose was also to evaluate the multiplicity of infection (MOI) of Lawsonia intracellularis in Sf9 cells at approximately 31 doses. Finally, the purpose was to compare Sf9 negative controls (uninfected cells) with Sf9 cells infected with Lawsonia intracellularis during biochemical and mass spectrometry analysis.

Materials and Methods.

| | | Part (item | |
|---|---|---|---|
| | Description | number) | Lot |
| Parent cell | Sf9 | na¹ | Pass 8 |
| Growth and Maintenance | Ex-Cell ™ 420 | 14420-1000M | 5E0184 |
| media (per variable) | | | |
| Comparator Growth and | IPL-41 | 17760 | 75K2370 |
| Maintenance media (per | | | |
| variable) | | | |
| Live Lawsonia | Titer: | na¹ | na¹ |
| intracellularis | 5.0 dose/ml | | |

¹na = not applicable

Cell and Media Information. The cell culture was Sf9 cells (Gibco® Cell Culture Systems, Invitrogen, Carlsbad, Calif., USA). The growth and maintenance media was Ex-Cell™ 420 Serum-Free Medium for Insect Cells with L-glutamine (JRH biosciences, Lenexa, Kans., USA; Catalog number 14420, item number 14420-1000M). The comparator growth and maintenance media was IPL-41 Insect Medium from Sigma-Aldrich Co., St. Louis, Mo., USA; Catalog number 17760; Lot number 75K2370). The seed culture contained modified live, non-virulent Lawsonia intracellularis bacteria.

Cell Numbers and Planting Information. Two stock solutions were used. The first was a 300-ml stock suspension maintained at 27° C. containing 3.18×10⁶ Sf9 cells per ml (viability of 91.4%) in IPL-41 media. The second was a 300-ml stock suspension maintained at 27° C. containing 1.78×10⁶ Sf9 cells per ml (viability of 97.8%) in Ex-Cell™ 420 media. Cells at six days of age were passed to 500-ml spinner flasks. A total of 179.8 ml of fresh Ex-Cell™ 420 media was put into spinner flask numbers 1 and 2, and 1.25×10⁸ cells (70.2 ml of the Ex-Cell™ 420 stock suspension) were planted into the media, resulting in approximately 250 ml total volume with 0.5×10⁶ cells/ml. A total of 210.7 ml of fresh IPL-41 media was put into spinner flask numbers 3 and 4, and 1.25×10⁸ cells (39.3 ml of the IPL-41 stock suspension) were planted into the media, resulting in approximately 250 ml total volume with 0.5×10⁶ cells/ml.

Variable Description.

| Vessel | Growth | | Seed | | Media |
|---|---|---|---|---|---|
| Number | Temperature | Description | (ml) | Doses | Supplement |
| 1 (Ex- | 27° C. | Pos. control | 6.25 | 31.25 | Day 7 |
| Cell ™ 420) | | | | | |
| 2 (Ex- | 27° C. | Neg. control | 0.00 | 0.00 | Day 7 |
| Cell ™ 420) | | | | | |
| 3 (IPL-41) | 27° C. | Pos. control | 6.25 | 31.25 | Day 7 |
| 4 (IPL-41) | 27° C. | Neg. control | 0.00 | 0.00 | Day 7 |

Vessel Configuration. All vessels were configured with one fixed-length drop tube to 80% depth and a two-port SST assembly configured with 0.1 μm sterile filters.

Process Parameters. Temperature was maintained at 27° C. for all vessels. Vessels were agitated at 100 rpm. Oxygen (O₂) levels were variable. pH levels were not monitored or controlled. When establishing the atmosphere in vessels 1 and 3, the vessels were sparged with a specialty gas comprising 10% hydrogen, 10% CO₂ and 80% nitrogen that was filtered through a 0.1 μm filter to prevent contamination. The sparge rate was 5-10 cc/second for one minute for 250 ml of media. The sparge rate was 5-10 cc/second for two minutes for 500 ml of media. To prevent diffusion, vessels were hemostat closed after gassing. Vessels 2 and 4, which were left at ambient conditions, possessed a 0.1 μm filter housing that was not hemostat closed. Hence, free gas exchange could occur under normal atmospheric conditions via the filter housing.

Infection. For Vessels 1 and 3, the Sf9 cells were infected when they were planted in the vessels (Day 0, Hour 0). Seed culture was introduced at a ratio of 1:40 of the vessel plant volume (i.e., 6.25 ml seed per 250 ml volume). Multiplicity of Infection (MOI) was not determined.

Media Supplementation. On Day 7 post planting of the Sf9 cells into the vessels, Vessels 1 and 2 were supplemented with 250 ml of Ex-Cell™ 420, and Vessels 3 and 4 were supplemented with IPL-41.

Harvest. Samples were taken on Days 0, 3, 4, 7 (presupplementation), 9, 11, and 14 post planting of the Sf9 cells into the vessels. After obtaining the Day 14 sample from the vessels, the remainder of the vessel contents was dispensed into large plastic vessels and frozen at minus 80° C.

Results. The SF9 cells grew significantly better in the Ex-Cell™ 420 media as compared with the IPL-41 media (See Table 13). The Lawsonia intracellularis was able to be cultured in both media (See Tables 14 and 15), but it achieved higher yields in the Ex-Cell™ 420 versus the IPL-41 media (212 fold vs 4.4 fold).

TABLE 13
Viable Sf9 Cell Counts per Vessel*

| | Vessel 1. | Vessel 2. | Vessel 3. | Vessel 4. |
|---|---|---|---|---|
| | Ex-Cell 420 | Ex-Cell 420 | IPL-41 | IPL-41 |
| | Positive | Negative | Positive | Negative |
| Day 0** | 1.3E+08 | 1.3E+08 | 1.3E+08 | 1.3E+08 |
| Day 3 | 6.2E+08 | 6.7E+08 | 2.15E+08 | 2.10E+08 |
| Day 4 | 5.8E+08 | 6.6E+08 | 2.10E+08 | 1.7E+08 |
| Day 7 presuppl | 7.3E+08 | 8.6E+08 | 1.66E+08 | 7.00E+07 |
| Day 9 | 5.92E+08 | 6.48E+08 | 1.04E+08 | 4.80E+07 |
| Day 11 | 6.40E+08 | 4.88E+08 | 7.90E+07 | 5.70E+07 |
| Day 14 | 6.00E+08 | 6.80E+08 | 5.20E+07 | 6.20E+07 |

*Data shown in scientific notation (e.g., 1.30E+08 = 1.30 × 10⁸)
**Number of days after planting of Sf9 cells into the vessels

TABLE 14
L. intracellularis Copies per Vessel (qRT-PCR)*

| | Vessel 1. | Vessel 2. | Vessel 3. | Vessel 4. |
|---|---|---|---|---|
| | Ex-Cell 420 | Ex-Cell 420 | IPL-41 | IPL-41 |
| | Positive | Negative | Positive | Negative |
| Day 0** | 1.60E+08 | | 1.80E+08 | |
| Day 3 | 4.97E+08 | | 2.70E+08 | |
| Day 4 | 4.25E+08 | | 8.00E+08 | |
| Day 7 presuppl | 4.50E+09 | | 3.00E+08 | |
| Day 7 postsuppl | 2.30E+09 | | 1.70E+08 | |
| Day 9 | 1.80E+10 | | 1.70E+08 | |
| Day 11 | 3.40E+10 | | 6.70E+07 | |
| Day 14 | 3.10E+10 | | 8.20E+07 | |

*Data shown in scientific notation (e.g., 1.60E+08 = 1.60 × 10⁸)
**Number of days after planting of Sf9 cells into the vessels

TABLE 15
Fold increase (qRT-PCR) in L. intracellularis

| | Vessel 1. | Vessel 2. | Vessel 3. | Vessel 4. |
|---|---|---|---|---|
| | Ex-Cell 420 | Ex-Cel 420 | IPL-41 Positive | IPL-41 Negative |
| | Positive Control | Negative Control | Control | Control |
| Day 0 to 7 presuppl* | 28.1 | | 1.7 | |
| Day 7 postsuppl to 14 | 13.5 | | 0.5 | |
| Day 0-14 | 193.8 | | 0.5 | |
| Maximum increase | 212.5 | | 4.4 | |

*Number of days after planting of Sf9 cells into the vessels

### Example 6

PPE propagation experiment in flasks varying density.

Purpose. The purpose of this experiment was to evaluate the growth of Lawsonia intracellularis using the Sf9 (spodoptera frugiperda) cell line planted with three different densities in an anchorage system.

Materials and Methods.

| | | Part (item | |
|---|---|---|---|
| | Description | number) | Lot |
| Parent cell | Sf9 | na¹ | Pass 20 |
| Growth media | Ex-Cell ™ 420 | 14420-1000 M | 5CO416 |
| Sera in the Growth Media | IFBS² | 12107-1000M | 3H0548 |
| Live Lawsonia | Titer: | na¹ | na¹ |
| intracellularis | 5 dose/100 ml | | |

¹na = not applicable
²IFBS = Irradiated Fetal Bovine Serum

Cell and Media Information. The cell culture was Sf9 cells (Gibco® Cell Culture Systems, Invitrogen, Carlsbad, Calif., USA). The growth and maintenance media was Ex-Cell™ 420 Serum-Free Medium for Insect Cells with L-glutamine containing 5% Fetal Bovine Serum Sourced in USA gamma irradiated by SER-TAIN™ Process (JRH Biosciences, Lenexa, Kans., USA; Catalog number 12107, item number 12107-1000M). The seed culture contained modified live, non-virulent Lawsonia intracellularis bacteria. The seed culture was a subaliquot of the original vaccine.

Cell Numbers and Planting Information.

| Vessel Number | Sf9 cell density* | Lawsonia inoculum |
|---|---|---|
| 1 | 2E+7 cells/75 cm² | 1.25 mls per 50 ml |
| | | maintenance (1:40) |
| 2 | 1E+7 cells/75 cm² | 1.25 mls per 50 ml |
| | | maintenance (1:40) |
| 3 | 5E+6 cells/75 cm² | 1.25 mls per 50 ml |
| | | maintenance (1:40) |
| 4 | 5E+6 cells/75 cm² | 1.25 mls per 50 ml |
| | | maintenance (1:40) |

*Data shown in scientific notation (e.g., 2E+07 = 2 × 10⁷)

Process Parameters. For all vessels, temperature was maintained at 27° C. Oxygen (O₂) levels were variable. pH levels were not monitored or controlled. When establishing the specialty gas atmosphere in Vessels 1 to 4, the 75 cm² flasks were placed in a BBL™ GasPak™ System (Becton, Dickinson and Company, Franklin Lakes, N.J., USA) and the vessel was sealed. The vessel was then vacuum evacuated and replenished with 10% CO₂, 10% H₂, and 80% N₂. To prevent diffusion, vessel was hemostat closed after gassing.

Infection. The Sf9 cells were infected less than 2 hours after they were planted in the vessels. Seed culture was introduced into the vessels at a ratio of 1:40 of the vessel plant volume (i.e., 1.25 ml seed per 50 ml volume). Multiplicity of Infection (MOI) was not determined.

Media Supplementation. The media was not supplemented during this experiment.

Harvest. Samples were taken on Days 0, 7, 10, and 13 post planting of the Sf9 cells into the vessels.

Results. Each of the SF9 cell densities evaluated yielded significant growth of Lawsonia intracellularis (See Table 16). The vessels planted with 2E+7, 1E+7, and 5E+7 cell densities yielded increases of 15.3, 24.5 and 27.9 fold, respectively.

TABLE 16
L intracellularis Copies per 1000 ul (qRT-PCR)*

| | Vessel 1. | Vessel 2. | Vessel 3. | Vessel 4. |
|---|---|---|---|---|
| | 2E+7 cells | 1E+7 cells | 5E+6 cells | 5E+6 cells |
| Day 0** | 1.70E+06 | 1.06E+06 | 1.04E+06 | 1.38E+06 |
| Day 7 | 8.20E+06 | 4.20E+06 | 3.80E+06 | 4.80E+06 |
| Day 10 | 2.40E+07 | 5.80E+06 | 1.32E+07 | 5.00E+06 |
| Day 13 | 2.60E+07 | 2.60E+07 | 2.90E+07 | |

*Data shown in scientific notation (e.g., 1.70E+06 = 1.70 × 10⁶)
**Number of days after planting of Sf9 cells into the vessels

### Example 7

PPE propagation experiment in avian cells varying atmospheric conditions.

Purpose. The purpose of this experiment was to evaluate the growth of Lawsonia intracellularis using the CEV-1 avian cell line at 37° centigrade (C.) under CO₂ versus a specialty gas atmospheric conditions.

Materials and Methods.

| | | Part (item | |
|---|---|---|---|
| | Description | number) | Lot |
| Parent cell | CEV-1 | na¹ | Pass 42 |
| Media | DMEM/F12 | 21041-025 | 1239862 |
| Sera in the Media | 10% IFBS² | 12107-1000M | 8129053 |
| Live Lawsonia | Titer: dose/ml 2.5 | na¹ | na¹ |
| intracellularis | dose/ml | | |

¹na = not applicable
²IFBS = Irradiated Fetal Bovine Serum

Cell and Media Information. The CEV-1 cells were obtained from stock cultures maintained at Pfizer, Inc. The growth and maintenance media were DMEM:F12 1:1 with L-Glutamine (Gibco® Cell Culture Systems, Invitrogen, Carlsbad, Calif., USA; Catalog number 21041-025) containing 10% Fetal Bovine Serum Sourced in USA gamma irradiated by SER-TAIN™ Process (JRH Biosciences, Lenexa, Kans., USA; Catalog number 12107, item number 12107-1000M). The seed culture contained modified live, non-virulent Lawsonia intracellularis bacteria.

Cell Numbers and Planting Information. A 20-ml stock suspension containing 2.7e6 of CEV-1 cells per ml was used. Parent cells (prior to passage) were 4 days old. Cells at 0 days of age were passed to 500-ml spinner flasks. A total of 240 ml of fresh media was put into each spinner flask, and 2.5e7 cells (10 ml of the stock suspension) were planted into the media, resulting in approximately 250 ml total volume with 100,000 cells/ml.

Variable Description.

| Vessel Number | Temperature | Atmosphere | Seed (ml) |
|---|---|---|---|
| 1 | 27° C. | Specialty gas | 12.5 |
| 2 | 27° C. | CO₂ | 12.5 |

Vessel Configuration. All vessels were configured with one fixed-length drop tube to 80% depth and a two-port SST assembly configured with 0.1 μm sterile filters.

Process Parameters. For both vessels, temperature was maintained at 37° C. Both vessels were agitated at 100 rpm. Oxygen (O₂) levels were variable. pH levels were not monitored or controlled. When establishing the specialty gas atmosphere in Vessel 1, the vessel was sparged with a specialty gas comprising 10% hydrogen, 10% CO₂ and 80% nitrogen that was filtered through a 0.1 μm filter to prevent contamination. The sparge rate was 5-10 cc/second for one minute for 250 ml of media. The sparge rate was 5-10 cc/second for two minutes for 500 ml of media. To prevent diffusion, vessel was hemostat closed after gassing. Vessel 2, which was maintained in a 5% CO₂ environment, possessed a 0.1 μm filter housing that was not hemostat closed. Hence, free gas exchange could occur with the 5% CO₂ environment via the filter housing.

Infection. The CEV-1 cells were infected 24 hours after they were planted in the vessels (Day 1). Seed culture was introduced into the vessels at a ratio of 1:20 of the vessel plant volume (i.e., 12.5 ml seed per 250 ml volume). Multiplicity of Infection (MOI) was not determined.

Media Supplementation. All vessels were supplemented with 250 ml of DMEM F12 10% IFBS on Day 8 post planting of the CEV-1 cells into the vessels.

Harvest. Samples were taken on Days 1, 4, 7, 8 (both pre- and post-supplementation), 9, 10, 11, and 14 post planting of the CEV-1 cells into the vessels.

Results. The CEV-1 cells grew under the conditions of this study as determined by cell density and viability (See Tables 17 and 18). Lawsonia intracellularis grew in the CEV-1 avian cells in an environment of 37° C. and speciality gas (microaerophilic) conditions (See Table 19). The CEV-1 cells maintained in the speciality gas yielded a 7.9 fold increase from Day 1-Day 14 post planting of the CEV-1 cells into the vessels. The culture maintained in the CO₂ environment did not yield an increase in Lawsonia intracellularis during this same period.

TABLE 17
Viable Cell Counts per Vessel
Viable CEV-1 Cell Counts per Vessel*

| | Vessel 1. | Vessel 2. |
|---|---|---|
| | CEV-1 37° C. | CEV-1 37° C. |
| | Specialty Gas | CO₂ |
| Day −1 | 2.5E+7 | 2.5E+7 |
| Day 0** | 9.0E+6 | 8.0E+6 |
| Day 3 | 4.5E+7 | 7.3E+7 |
| Day 6 | 1.2E+7 | 2.1E+7 |
| Day 7*** | 7.0E+6 | 1.3E+7 |
| Day 8 | 1.2E+7 | 1.4E+7 |
| Day 9 | 8.0E+6 | 2.1E+7 |
| Day 10 | 9.0E+6 | 4.3E+7 |
| Day 13 | 7.0E+6 | 8.6E+7 |

*Data shown in scientific notation (e.g., 1.3E+8 = 1.3 × 10⁸)
**Date of infection
***Date of media supplementation

TABLE 18
Cell Viability by Vessel (Percent)
Viable CEV-1 Viability (Percent) per Vessel

| | Vessel 1. | Vessel 2. |
|---|---|---|
| | CEV-1 37° C. | CEV-1 37° C. |
| | Specialty Gas | CO₂ |
| Day −1 | 100.0% | 100.0% |
| Day 0* | 96.9% | 96.6% |
| Day 3 | 90.0% | 100.0% |
| Day 6 | 41.4% | 47.7% |
| Day 7*** | 29.2% | 39.4% |
| Day 8 | 41.4% | 34.1% |
| Day 9 | 30.8% | 63.6% |
| Day 10 | 47.4% | 78.2% |
| Day 13 | 29.2% | 53.4% |

*As determined by trypan blue dye exclusion
**Date of infection
***Date of media supplementation

TABLE 19
L. intracellularis Copies per Vessel (qRT-PCR)*

| | Vessel 1. | Vessel 2. |
|---|---|---|
| | Specialty Gas | CO₂ |
| Day 1** | 1.90E+09 | 2.20E+09 |
| Day 4 | 1.75E+09 | 1.45E+09 |
| Day 7 | 3.35E+09 | 1.40E+09 |
| Day 8 Pre suppl | 2.20E+09 | 1.35E+09 |
| Day 8 Post suppl | 2.70E+09 | 1.70E+09 |
| Day 9 | 3.30E+09 | 1.90E+09 |
| Day 10 | 3.70E+09 | 2.10E+09 |
| Day 11 | 4.50E+09 | 1.90E+09 |
| Day 14 | 1.50E+10 | 1.70E+09 |

*Data shown in scientific notation (e.g., 1.90E+09 = 1.0 × 10⁹)
**Number of days after planting of CEV-1 cells into the vessels

All of the methods disclosed and claimed herein can be made and executed without undue experimentation in light of the present disclosure. While the methods of this invention have been described in terms of different embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### Example 8

Culture of Lawsonia intracellularis in Sf-21 Insect Cells

Materials and Methods. A bottle of Graces Insect Cell media (Gibco cat no. 11605-094) was warmed to 27° C. for thirty minutes prior to use. A 1 ml cryovial containing 10⁶ Sf-21 cells per ml was obtained was obtained and thawed for 15 minutes at 37° C., to ensure all frozen suspension was gone. A 10% fetal bovine serum (FBS; JRH cat no. 12103-500M) plus 1% L-glutamine (L-glut; Gibco cat no. 25030-081) in Graces media for cell suspension.

Once the cryovial of cells was thawed, the contents were resuspended in 10 ml of the 10% FBS, 1% L-glut in Graces and centrifuged at 800 rpm for 5 minutes to remove DMSO freezing solution from the cells. When completed, the supernatant solution was removed and discarded before the cells were gently resuspended in 10 mls of 10% FBS, 1% L-glut in Graces.

All ten mls of resuspended cells were transferred to a Corning T-75 cm² flask (cat no. 430641; vent cap) and an additional 5 mls of 10% FBS, 1% L-glut in Graces was added to bring the volume to 15 mls in the culture flask. The flask was incubated for 1 hour at 27° C. and then completely refed with 15 mls of 10% FBS, 1% L-glut in Graces. The process was meant to remove any dead or unattached Sf-21 cells. This process is repeated once more one hour after the first flask refeeding. The resulting monolayer was approximately 40-45% by days end.

48 hours was allowed for the Sf-21 cells to reach log phase growth (80-95% confluent monolayer). At this time cells were gently washed into the supernatant media. The media and cells were centrifuged at 1,000 g for 5 minutes following which, the supernatant media was discarded and the cells gently resuspended into 5 mls of 10% FBS, 1% L-glut in Graces for further propagation.

The resulting cell suspension was diluted into 25 ml of 10% FBS, 1% L-glut in Graces media and split into 2 T-75 cm² culture flasks. The flasks were again incubated for one hour at 27° C. and then refed with the same media preparation as before to again remove any nonviable or dead cells. The process was repeated again one hour later and the cells were allowed to incubate unimpeded for at least 4 hours prior to infection.

One cryovial containing supernantant Lawsonia intracellularis (10⁵-10⁶ Li/ml) was thawed at 37° C. per T-75 cm² flask to be infected. One flask was kept for cell propagation and the other was used for infection with Li.

Once the cryovial(s) was thawed completely, it was added to the approximately 20-30% confluent monolayer of Sf-21 cells. The preferred infection point was 4-6 hours following the second refeeding of the uninfected Sf-21 cells.

Each infected flask was evacuated to 500 mmHg and gassed with 100% H₂ for approximately 30 seconds prior to transfer to 27° C. incubator. The dividing cycle of the Sf-21 insect cell is approximately 48-60 hours, and as a result, cultures were propagated or terminated at such time. Somewhere around 40-42 hours post-infection, the Sf-21 cell monolayer was scraped and stained following IPX (monoclonal or polyclonal) staining technique to evaluate the percent infection of the Sf-21 cells.

### Example 9

Culture of Lawsonia intracellularis in Sf-21 Insect Cells adapted to monolayer growth.

Materials and Methods. About 10⁶ Sf-21 cells was added to ten mls of Graces Insect Cell media (Gibco cat no. 11605-094) with 10% fetal bovinee serum (FBS; JRH cat no. 12103-500M) plus 1% L-glutamine (L-glut; Gibco cat no. 25030-081). All ten mls of resuspended cells were transferred to a Corning T-75 cm² flask (cat no. 430641; vent cap) and an additional 5 mls of 10% FBS, 1% L-glut in Graces was added to bring the volume to 15 mls in the culture flask. The flask was incubated for 1 hour at 27° C. and then completely refed with 15 mls of 10% FBS, 1% L-glut in Graces to remove any dead or unattached Sf-21 cells.

48 hours was allowed for the Sf-21 cells to reach log phase growth (80-95% confluent monolayer). At this time cells were gently washed into the supernatant media. The media and cells were centrifuged at 1,000 g for 5 minutes following which, the supernatant media was discarded and the cells gently resuspended into 5 mls of 10% FBS, 1% L-glut in Graces for further propagation.

The resulting cell suspension was diluted into 25 ml of 10% FBS, 1% L-glut in Graces media and split into 2 T-75 cm² culture flasks. The flasks were again incubated for one hour at 27° C. and then refed with the same media preparation as before to again remove any nonviable or dead cells. The process was repeated again one hour later and the cells were allowed to incubate unimpeded for at least 4 hours prior to infection.

One cryovial containing supernantant Lawsonia intracellularis (10⁵ Li/ml) was thawed at 37° C. per T-75 cm² flask to be infected. Once the cryovial(s) was thawed completely, it was added to the approximately 20-30% confluent monolayer of Sf-21 cells. The preferred infection point was 4-6 hours following the second refeeding of the uninfected Sf-21 cells.

Each infected flask was evacuated to 500 mmHg and gassed with 100% H₂ for approximately 30 seconds prior to transfer to a 27° C. incubator. Around 40-42 hours post-infection, the Sf-21 cell monolayer was scraped and stained following IPX (monoclonal or polyclonal) staining technique to evaluate the percent infection of the Sf-21 cells.

Results. About 10 to 15% of the Sf-21 monolayer was infected with more than 30 Li per cell, yielding about 10⁶ to 10⁷ Li per T-75 flask.

What is claimed is:

1. A method for growing Lawsonia intracellularis in non-mammalian cells comprising
a. planting the cells in a vessel containing a suitable media;
b. inoculating the cells with L. intracellularis;
c. growing the inoculated cells; and
d. harvesting the L. intracellularis.

2. The method of claim 1, wherein the cells are selected from the group consisting of insect cells, Schneider cells, and avian cells.

3. The method of claim 2, wherein said insect cells are selected from Sf9 cells, SF21 cells, SF+ cells, Hi-Five cells, or insect larval cells.

4. The method of claim 3, wherein the cells are Sf9 insect cells.

5. The method of claim 2, wherein said avian cells are selected from CEV-1 cells or avian embryo cells.

6. The method of claim 1, wherein the media is free of animal protein.

7. The method of claim 1, wherein the media comprises an animal protein.

8. The method of claim 1, wherein said growing is performed at a temperature of about 20° C. to about 39° C.

9. The method of claim 1, wherein said cells are insect cells and the growing is at a temperature of about 25° C. to about 29° C.

10. The method of claim 1, wherein said cells are avian cells and the growing is at a temperature of about 35° C. to about 39° C.

11. The method of claim 1, wherein the vessel contains microaerophilic or aerophilic conditions.

12. The method of claim 11, wherein the microaerophilic conditions comprise a mixture of gasses of about 10% hydrogen, about 10% CO₂ and about 80% nitrogen.

13. The method of claim 1, wherein the multiplicity of infection (MOI) is from about 0.000001 to about 10 measured by quantitative Reverse Transcriptase Polymerase Chain Reaction (qRT-PCR).

14. The method of claim 1, wherein the MOI is from about 0.0001 to about 10 using qRT-PCR.

15. The method of claim 1, wherein the L. intracellularis is harvested from about 5 to about 25 days after inoculating the cells with L. intracellularis.

16. The method of claim 1, wherein the L. intracellularis is harvested from about 9 to about 15 days after inoculating the cells with L. intracellularis.

17. The method of claim 16, wherein the cells are planted in a density of about 100,000 to about 10,000,000 cells per ml.

18. The method of claim 16, wherein the cells are planted in a density of about 500,000 cells per ml to about 1,500,000 cells per ml.

19. The method of claim 16, wherein the media is free of animal protein.

20. The method of claim 19, wherein the cells are planted in a density of about 10,000 to about 1,000,000.

21. The method of claim 19, wherein the cells are planted in a density of about 60,000 to about 250,000 cells per cm².

22. The method of claim 19, wherein the media comprises an animal protein.

23. The method of claim 22, wherein the animal protein is present in a concentration from about 0.5% to about 10%.

24. The method of claim 1, wherein the inoculated cells are grown in a media at a volume of at least 2 to 3 liters.

25. The method of claim 24, wherein the inoculated cells are grown in a media at a volume of at least 100 liters.
